(19) United States
(12) Patent Application Publication
(10) Pub. No.: US 2010/0055689 A1
(43) Pub. Date: Mar. 4, 2010

(54) MULTIFACTORIAL METHODS FOR DETECTING LUNG DISORDERS

(76) Inventors: Avrum Spira, Newton, MA (US); Marc E. Lenburg, Berkeley, CA (US); Jennifer E. Beane-Ebel, Rio Rancho, NM (US); Daniel Rippy, Sudbury, MA (US)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/414,555

(22) Filed: Mar. 30, 2009

Related U.S. Application Data
(60) Provisional application No. 61/040,434, filed on Mar. 28, 2008.

(51) Int. Cl. C12Q 1/68; G01N 33/53

(52) U.S. Cl. 435/6; 435/7.1

(57) ABSTRACT

Described herein are multifactorial methods for detecting, diagnosing or aiding in the diagnosis of lung disorders or disease, e.g., lung cancer. The methods disclosed utilize multiple diagnostic paradigms, for example, to improve diagnostic sensitivity, specificity, negative predictive value and/or positive predictive value over each of the paradigms alone. For example, a clinicogenomic model is disclosed for lung cancer diagnosis which combines clinical factors and gene expression, particularly a sensitive and specific gene expression biomarker.

### RELATED APPLICATIONS

This application claims the benefit of U.S. Provisional Application Ser. No. 61/040,434, filed Mar. 28, 2008, the entire teachings of which are incorporated herein by reference.

### GOVERNMENT SUPPORT

The invention was supported, in whole or in part, by grants R21CA106506 and R01CA124640 from the National Institutes of Health (National Cancer Institute). The U.S. Government has certain rights in the invention.

### BACKGROUND OF THE INVENTION

Lung cancer is the leading cause of cancer death due, in part, to lack of early diagnostic tools. Smokers are often suspected of having lung cancer based on abnormal radiographic findings and/or symptoms that are not specific for lung cancer. Fiberoptic bronchoscopy represents a relatively noninvasive initial diagnostic test in smokers with suspect disease, allowing cytologic examination of materials obtained via endobronchial brushings, bronchoalveolar lavage, and endobronchial and transbronchial biopsies of the suspect area. Unfortunately this method has relatively low sensitivity. Additional and more invasive diagnostic tests are routinely needed, increasing cost, incurring risk, and prolonging the diagnostic evaluation of patients with suspect lung cancer. Determining which suspect lung cancer patients with cancer-negative bronchoscopies should undergo these additional diagnostic tests is currently a matter of clinical judgment. Thus, it would be beneficial to have additional diagnostic tools which provide improved sensitivity and positive and negative predictive value for diagnosis of lung disease such as lung cancer.

### SUMMARY OF THE INVENTION

The invention described herein relates to multifactorial methods for detecting, diagnosing or aiding in the diagnosis of lung disorders or disease, e.g., lung cancer. The methods of the invention utilize multiple (i.e., two or more) diagnostic paradigms, for example, to improve diagnostic sensitivity, specificity, negative predictive value and/or positive predictive value over each of the paradigms alone. In preferred embodiments the diagnostic paradigms are independent of one another.

For example, in one embodiment the invention relates to a clinicogenomic model for lung cancer diagnosis which combines clinical factors and gene expression, particularly a sensitive and specific gene expression biomarker. Work described herein analyzed the likelihood of cancer in a set of smokers undergoing bronchoscopy for suspicion of lung cancer using the gene expression biomarker, clinical factors, and a combination of these data (the clinicogenomic model). A significant difference in performance of the clinicogenomic model was identified relative to the clinical factors alone. Indeed, the clinicogenomic model increases sensitivity and negative predictive value to 100% and results in higher specificity and positive predictive value compared with the other models. Accordingly, the use of the clinicogenomic model may expedite more invasive testing and definitive therapy for individuals with lung cancer, as well as reduce invasive diagnostic procedures for individuals without lung cancer.

In one embodiment the invention relates to a method of aiding in the diagnosis of lung disease in a patient suspected of having lung disease, comprising: analyzing two or more independent lung cancer-relevant diagnostic paradigms in a patient to be assessed; and determining a composite classification of the patient as having lung disease or not having lung disease. In one embodiment the lung disease is lung cancer. In one aspect the patient is a smoker or former smoker. In another aspect the patient has had an abnormal radiographic finding or a nondiagnostic bronchoscopy.

In a preferred embodiment the invention relates to a method wherein the two or more lung cancer-relevant diagnostic paradigms are selected from the group consisting of analyzing expression of one or more lung cancer-relevant genes in the patient, analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs. In another aspect the two or more lung cancer-relevant diagnostic paradigms comprise analyzing expression of one or more lung cancer-relevant genes in the patient. In one embodiment the two or more lung cancer-relevant diagnostic paradigms comprise analyzing expression of one or more lung cancer-relevant genes in the patient and one or more lung cancer-relevant diagnostic paradigms selected from the group consisting of analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs. In particular embodiments, the one or more lung cancer-relevant genes are all or a subset of the genes for which expression data is contained in Gene Expression Omnibus accession no. GSE4115. In a particular embodiment the two or more lung cancer-relevant diagnostic paradigms comprise analyzing expression of one or more lung cancer-relevant genes in the patient and analyzing one or more lung cancer-relevant clinical factors or variables of the patient.

The invention also relates to a method of determining a follow up treatment regimen for a patient suspected of having lung cancer, comprising analyzing two or more independent lung cancer-relevant diagnostic paradigms in a patient to be assessed, and classifying the patient as having cancer or not having cancer on the basis of the analysis, wherein a patient classified as having cancer is selected for invasive testing and/or commencement of therapeutic regimen, and a patient classified as not having cancer is monitored without invasive testing or commencement of therapeutic regimen.

In one embodiment the patient is a smoker or former smoker. In another embodiment the patient has had an abnormal radiographic finding or a nondiagnostic bronchoscopy. In a particular embodiment the two or more lung cancer-relevant diagnostic paradigms are selected from the group consisting of analyzing expression of one or more lung cancer-relevant genes in the patient, analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs. In another embodiment the two or more lung cancer-relevant diagnostic paradigms comprise analyzing expression of one or more lung cancer-relevant genes in the patient. In one aspect of the invention the two or more lung cancer-relevant diagnostic paradigms comprise analyzing expression of one or more lung cancer-relevant genes in the patient and one or more lung cancer-relevant diagnostic paradigms selected from the group consisting of analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs. In a particular aspect of the invention the one or more lung cancer-relevant genes are all or a subset of the genes for which expression data is contained in Gene Expression Omnibus accession no. GSE4115. In another embodiment of the invention the two or more lung cancer-relevant diagnostic paradigms comprise analyzing expression of one or more lung cancer-relevant genes in the patient and analyzing one or more lung cancer-relevant clinical factors or variables of the patient.

The invention also relates to a method of aiding in the diagnosis of lung cancer in a patient suspected of having lung cancer, comprising: obtaining a biological sample from the patient and analyzing expression of one or more lung cancer-relevant genes in the sample, wherein the one or more lung cancer-relevant genes are all or a subset of the genes for which expression data is contained in Gene Expression Omnibus accession no. GSE4115; analyzing one or more lung cancer-relevant clinical factors or variables of the patient; and determining a composite classification of the patient as having cancer or not having cancer.

In preferred embodiments the two or more lung cancer relevant diagnostic paradigms provide more specificity, positive predictive value, negative predictive value and/or sensitivity than at least one of the two or more paradigms alone (e.g., more than any of the two or more paradigms alone).

### BRIEF DESCRIPTION OF THE DRAWINGS

FIGS. 1A and 1B show the training and test sample sets used in the examples. The training and test samples were derived from a previously published study assaying airway epithelial gene expression from current and former smokers undergoing bronchoscopy for the clinical suspicion of lung cancer. FIG. 1A shows a gene expression biomarker previously constructed that predicts the presence of lung cancer using a training set of 77 patients. For the study described in the example one of these samples was removed due to incomplete smoking history, resulting in the logistic regression models being trained with data from 76 patients. The models were subsequently tested on the subset of training samples (n=56) that had cytopathology that was nondiagnostic of lung cancer. As shown in FIG. 1B, the biomarker was also tested on the subset of independent samples with nondiagnostic cytopathology (n=62) from the combined test and prospective validation sample sets (n=87) used in the previous study.

FIG. 2A-2C show ROC curves for the clinical model and the clinicogenomic model across the different sample sets. The clinical model (red line) includes the following variables: age, mass size, and lymphadenopathy; the clinical and biomarker model (black line) includes the above variables and the biomarker score. Both models were derived using the training set samples (n=76). FIG. 2A shows the ROC analysis of the nondiagnostic training set samples (n=56). The area under the curve for the clinical and clinicogenomic model is 0.84 and 0.90, respectively. FIG. 2B shows the ROC analysis of the test samples (n=62). The area under the curve for the clinical and clinicogenomic model is 0.94 and 0.97, respectively. FIG. 2C shows the ROC analysis of the combined training and test sets (n=118). The area under the curve for the clinical and clinicogenomic model is 0.89 and 0.94, respectively, which represents a significant difference between the two curves (P<0.05).

FIG. 3A-3C shows the performance of three logistic regression models across the test set samples. Samples with model-derived probabilities of having lung cancer ≧0.5 were classified as cancer, and samples with probabilities <0.5 were classified as noncancer. Orange, samples with a final diagnosis of cancer; blue, samples with a final diagnosis of no cancer. The saturation of the colors is representative of the proportion of each final diagnosis group classified as having cancer or no cancer by each of the models. For each model, the sensitivity (Sens), specificity (Spec), positive predictive value (PPV), and the negative predictive value (NPV) are shown. FIG. 3A shows the clinical model, FIG. 3B shows the biomarker model, and FIG. 3C shows the clinicogenomic model. The clinical model and the biomarker model each perform similarly with accuracies of 84% and 87%, respectively. The clinicogenomic model has a greater accuracy (94%), specificity, and positive predictive value than either of the other two models.

FIG. 4 shows the association between the probability of having lung cancer as predicted by the clinical model and physician's subjective assessment across the test set samples (n=62). The model-derived probabilities are shown on the y-axis, and the subjective clinical assessment on the x-axis. Red circles, complete agreement among three clinicians; black circles, agreement between two clinicians; green circles, no agreement. There are significant differences (P<0.01, Wilcoxon test) between the probabilities in the low versus medium group, the medium versus high group, and the low versus high group. Cancer status of each subject stratified by subjective risk assessment is shown in FIG. 5.

FIG. 5 shows the clinicogenomic model-derived lung cancer predictions stratified by cancer status and the physician's subjective assessment across the test set samples (n=62). Dark gray, a final diagnosis of cancer; light gray, a final diagnosis of non-cancer. Squares, correct clinicogenomic model predictions; circles, incorrect model predictions. Each of the samples classified as having a medium risk of lung cancer by physicians was correctly predicted by the clinicogenomic model.

FIG. 6 shows the demographic and clinical characteristics as well as the mean and SD for the biomarker scores stratified by cancer status and membership in the training or test sets (Table 1).

FIG. 7 shows information about the cell type, stage, and location of the tumors in the cancer patients, as well as the fraction of diagnostic bronchoscopies for each subgroup (Table 2).

FIG. 8 shows effect estimates and derived odds ratios for the variables in each of the three logistic regression models (Table 3).

FIG. 9 shows that the clinicogenomic model also accurately predicted lesions with a mass size <3 cm as well as poorly defined radiographic infiltrates in the test set (Table 4).

### DETAILED DESCRIPTION OF THE INVENTION

The invention described herein relates to multifactorial methods for detecting, diagnosing or aiding in the diagnosis of lung disorders or disease, e.g., lung cancer. The methods of the invention utilize multiple (i.e., two or more) diagnostic paradigms, for example, to improve diagnostic sensitivity, specificity, negative predictive value and/or positive predictive value over each of the paradigms alone. This is a particularly powerful approach where the predictions made under each paradigm used in the multifactorial method are independent of one another. The methods of the invention are of particular use in assessing subjects (patients) suspected of having a lung disorder (e.g., lung cancer) but who have cancer-negative bronchoscopies, but the methods may be beneficially utilized in diagnosing any patient suspected of having lung cancer or other lung disorder.

Paradigms useful in the invention include, but are not limited to, expression of one or more cancer-relevant genes, presence or absence or severity of one or more cancer-relevant clinical factors or variables, presence or absence of one or more cancer-relevant antibodies in the subject's blood, presence or absence of one or more cancer-relevant proteins in the subject's blood, and expression of one or more cancer-relevant microRNAs. Many specific methods of measuring gene expression (e.g., assays using probes and primers, microarrays, etc.), presence or absence of proteins and presence or absence of antibodies are well known in the art. Moreover, methods of measuring lung cancer-relevant clinical variables are also known in the art.

“Cancer-relevant” as used herein is intended to mean “associated with the presence or absence of cancer.” For example, a cancer-relevant gene is a gene differentially expressed (e.g., in timing, level or location (e.g., tissue or cell type)) in an individual with cancer as compared with an individual without cancer. In particular embodiments the cancer-relevant entities are lung cancer-relevant entities. As described herein, multifactorial methods may, without limitation, utilize two or more paradigms, three or more paradigms, four or more paradigms, etc.

One exemplary embodiment of the invention is described below. This embodiment utilizes a specific set of gene expression data (i.e., gene expression profiles from a specific set of lung cancer-relevant genes; a gene expression biomarker) and a specific set of lung cancer-relevant clinical factors. However, it should be clear that the invention is not limited to either these specific clinical factors or the specific set of genes from which the gene expression data was derived. Moreover, the invention is not limited to the use of these two particular paradigms (gene expression profiles and clinical factors).

For example, subsets of either parameter are intended to be encompassed by the invention, including subsets used in combination with other similar data or factors. In one embodiment, all or a subset of expression data contained in Gene Expression Omnibus accession no. GSE4115 can be used in combination with all or a subset of the clinical factors disclosed in the exemplary embodiment. Moreover, all or a subset of the gene expression data and/or the clinical factors used in the exemplary embodiment can be used in combination with additional gene expression data and/or clinical factors known in the art.

In some embodiments, different gene expression profiles (i.e., not the expression data contained in Gene Expression Omnibus accession no. GSE4115) known in the art to be relevant to the detection of lung disorders can be combined with all or a subset of the clinical factors disclosed in the exemplary embodiment to detect lung disease. In other embodiments, different clinical factors (i.e., not the set of clinical factors disclosed in the exemplary embodiment) known in the art to be relevant to the detection of lung disorders can be combined with all or a subset of the gene expression profiles disclosed in the exemplary embodiment.

In further embodiments, different gene expression profiles (i.e., not the expression data contained in Gene Expression Omnibus accession no. GSE4115; determined from different genes) known in the art to be relevant to the detection of lung disorders can be combined with different clinical factors (i.e., not the set of clinical factors disclosed in the exemplary embodiment) known in the art to be relevant to the detection of lung disorders. The methods and algorithms described in the exemplary embodiment can be used with the data obtained from any of the paradigms, e.g., any lung cancer-relevant biomarkers as or any clinical factors, to predict or detect disorders of the lung. These methods and algorithms may be optimized to give greater weight to the paradigm(s) having greater predictive value and lesser weight to the paradigm(s) having lower predictive value.

For example, alternative gene expression biomarkers for use in the invention can be found in U.S. Patent publication 2007-0148650, U.S. Patent publication 2006-0154278, U.S. patent application Ser. No. 11/918,588 (filed Oct. 15, 2007), U.S. Provisional Application Ser. No. 60/994,637 (filed Sep. 19, 2007), U.S. Provisional Application Ser. No. 60/994,643 (filed Sep. 19, 2007) and PCT Publication WO07/103541. The teachings of all of these patent applications are incorporated herein by reference in their entirety. Additional gene expression biomarkers relevant to the diagnosis of lung disorders are also known in the art.

Clinical factors for use in the invention include, but are not limited to, all clinical factors described in the exemplary embodiment, whether used in the clinicogenomic diagnostic trial conducted as described or not. Particular clinical factors for use in the invention include, but are not limited to, age, smoking history (including number of pack-years, age started, intensity of smoking and years since quitting), history of asbestos exposure, clinical symptoms including hemoptysis and weight loss, size of nodule or mass and radiographic appearance on chest imaging, presence of lymphadenopathy, clinical or radiographic evidence for metastatic disease, evidence of airflow obstruction on spirometry, uptake of fluorodeoxyglucose on positron emission tomography scan, exposure to any known or suspected carcinogen, the type of tobacco product used by the subject, the presence or absence of chest pain in the subject, presence or absence of shortness of breath in the subject, presence or absence of episodic shortness of breath in the subject, presence or absence of blood in the sputum of the subject, presence or absence of a cough in the subject, presence or absence of an episodic cough in the subject, presence, absence or amount in the blood of the subject of one or more antibodies associated with lung cancer (e.g., Zhong et al., Am. J. Respr Crit Care Med 172:1308-1314 (2005); Zhong et. al., J Thorac Oncol 1:513-519 (2006)), and combinations thereof. It is envisioned that the clinical factors may be scored on the basis of presence or absence or may be scaled on the basis of severity or frequency.

In another embodiment of the invention the multifactorial diagnostic method utilizes presence, absence or amount in the blood of the subject of one or more antibodies associated with lung cancer (see e.g., Zhong et al., Am. J. Respr Crit Care Med 172:1308-1314 (2005); Zhong et al., J Thorac Oncol 1:513-519 (2006)) along with gene expression data (to produce an immunogenomic diagnostic) or along with clinical variables (to produce an immunoclinico diagnostic) or in combination with both gene expression data and clinical variables (to produce an immunoclinicogenomic diagnostic). In some embodiments of the invention the method utilizes the presence, absence or amount in the blood of the subject of one or more cancer-relevant antibodies along with one or more additional diagnostic paradigms.

In other embodiments of the invention the multifactorial diagnostic method utilizes the presence, absence or amount in the blood of the subject of one or more cancer-relevant proteins. Cancer-relevant proteins include, but are not limited to, human aspartyl beta-hydroxylase (HAAH), carcinoembryonic antigen (CEA), retinol binding protein (RBP), alpha-1-antitrypsin (AAT), squamous cell cardinoma antigen (SCCA), serum amyloid A, and tumor-associated NADH oxidase (tNOX). In some embodiments of the invention the method utilizes the presence, absence or amount in the blood of the subject of one or more cancer-relevant proteins along with one or more additional diagnostic paradigms.

In other embodiments of the invention the multifactorial diagnostic method utilizes expression of one or more cancer-relevant microRNAs along with one or more additional diagnostic paradigms. For example, microRNAs (miRNAs) which are differentially expressed in smokers and non-smokers have been described (Schembri et al., Proc Natl Acad Sci USA 106:2319-2324 (2009)). In one embodiment the one or more lung cancer-relevant miRNAs are selected from the group consisting of miR-337, miR-18a, miR-189, miR-365, miR-181d, miR-10b, miR-150, miR-218, miR-338, miP-362, miR-17-3p, miR-15a, miR-652, miR-106b, miR-19b, miR-106a, miR-128a, miR-30a-3p, miR-128b, miR-130a, miR-500, miR-363, miR-199b, miR-223, miR-625, miR-99a, miR-125b, and miR-146a. In a particular embodiment the miRNA is one or more of miR-218, miR-128b, miR-500 and miR-181d.

Lung cancer-relevant diagnostic paradigms which are independent are preferably used in combination as described herein to improve sensitivity, specificity, positive predictive value and/or negative predictive value of the paradigms individually. Suitable combinations of paradigms may improve all or a subset of sensitivity, specificity, positive predictive value and/or negative predictive value. Particular paradigms may be known to be independent in the art; alternatively sets of paradigms can be assessed as described below to determine their independence from one another.

In the context of the invention, diagnostic calls (e.g., cancer/noncancer) are made in each of the paradigms as they are made in the art. For example, a gene expression profile of one or more cancer-relevant genes is obtained from a biological sample of a patient to be assessed, and the expression profile is compared to a control or standard to determine whether the patient has or doesn't have cancer on the basis of that gene expression profile. The diagnostic calls from each of the utilized paradigms are combined to produce an overall score or classification to produce a multifactorial diagnostic call or classification. Statistical methods for each of these steps are described herein, and others are known in the art.

A previous study identified a gene expression biomarker capable of distinguishing cytologically normal large airway epithelial cells from smokers with and without lung cancer (Spira et al., Nat Med 13:361-366 (2007)). These cells can be collected in a relatively noninvasive manner from bronchial airway brushings of patients undergoing bronchoscopy for the suspicion of lung cancer. The cytopathology of cells obtained during bronchoscopy is 100% specific for lung cancer, but has a limited sensitivity of between 30% and 80%, depending on the stage and location of the cancer, with early-stage disease and peripheral cancers having the lowest sensitivity (Schreiber and McCrory, Chest 123:115-28S (2003)).

As a result, physicians are confronted with a difficult decision on how to manage the care of patients with potentially early-stage curable disease, when bronchoscopy does not return any cells with aberrant cytopathology. Often the decision about whether to proceed with more sensitive and often more invasive diagnostic procedures or to determine if the initial suspicious radiographic finding resolves in subsequent repeat imaging studies is based on a subjective assessment of the patient's clinical and radiographic risk factors for lung cancer. As the large airway gene expression biomarker uses material that can be easily collected at the time of bronchoscopy (prolonging the procedure by only 2-3 additional minutes), this test could be a useful component of the decision-making process if the biomarker captures information about lung cancer risk that is otherwise occult.

The results described herein suggest that the pattern of gene expression in large airway epithelial cells reflects information about the presence of lung cancer that is independent of other clinical risk factors. This interpretation results from a comparison of models that contain either clinical variables or the biomarker with a combined clinicogenomic model. The comparison shows that the biomarker is significantly associated with the probability of having lung cancer in both the biomarker and clinicogenomic models and that the importance of each of the variables in the combined clinicogenomic model is similar to their importance in the initial uncombined models.

The clinicogenomic model is a better predictor of lung cancer than either of the initial models in an independent test set. ROC curve analysis shows that the clinicogenomic model performs significantly better than the clinical model. Furthermore, the clinicogenomic model increases the sensitivity, specificity, positive predictive value and negative predictive value of the clinical model, and its accuracy does not seem to be influenced by the size or location of the lesion.

Despite the limitations of a small sample size and limited clinical parameters, it is encouraging that subjective clinical assessment based on a patient's complete medical record is associated with the clinical model probabilities. This is particular important given that certain variables, such as positron emission tomography scan findings, were not included in the clinical model because these studies were done on only a small number of the subjects in the cohort. All available data, such as positron emission tomography scan findings, were, however, considered by the pulmonary physicians as part of their subjective assessment of lung cancer likelihood. Further, the clinicogenomic model seems to correctly classify patients assigned to the medium risk subgroup by the clinical subjective assessment. This subgroup of patients is one that is likely to be especially challenging to manage clinically, as almost a third of these patients went on to have a final diagnosis of lung cancer.

The data disclosed herein suggest that a clinicogenomic model that combines gene expression with clinical risk factors for lung cancer can serve to identify those patients who would benefit from further invasive testing (e.g., lung biopsy) to confirm the presumptive lung cancer diagnosis and thereby expedite the diagnosis and treatment for their underlying malignancy. In addition, use of the clinicogenomic diagnostic may result in a reduction in the number of individuals without lung cancer who are subjected to additional and more invasive procedures to rule out a lung cancer diagnosis following a nondiagnostic bronchoscopy. Clinicians could more confidently use less invasive and less costly approaches (e.g., repeat computed tomography scan in 3-6 months) to follow-up patients with a low clinicogenomic lung cancer risk score.

The ability of gene expression profiles within cytologically normal airway epithelium to serve as a biomarker for lung cancer raises questions about the underlying biology of the cancer-specific molecular changes observed in these cells. The high diagnostic accuracy for the biomarker in the setting of small peripheral lung lesions suggests that changes in airway gene expression between smokers with and without lung cancer are unlikely to be a direct effect of the tumor. The presence of antioxidant and inflammation-related genes in the gene expression biomarker raises the possibility that the biomarker detects an airway-wide cancer-specific difference in response to tobacco smoke exposure. Thus, alternations in gene expression could precede the development of lung cancer and explain the somewhat lower specificity of the biomarker relative to its sensitivity. If this is true, the biomarker might potentially be a useful tool to identify smokers at highest risk for disease who may benefit from chemopreventive strategies.

The invention will be further described by the following non-limiting embodiment. The teachings of all cited references are incorporated herein by reference in their entirety.

### Examples

### Materials and Methods

### Patient Population

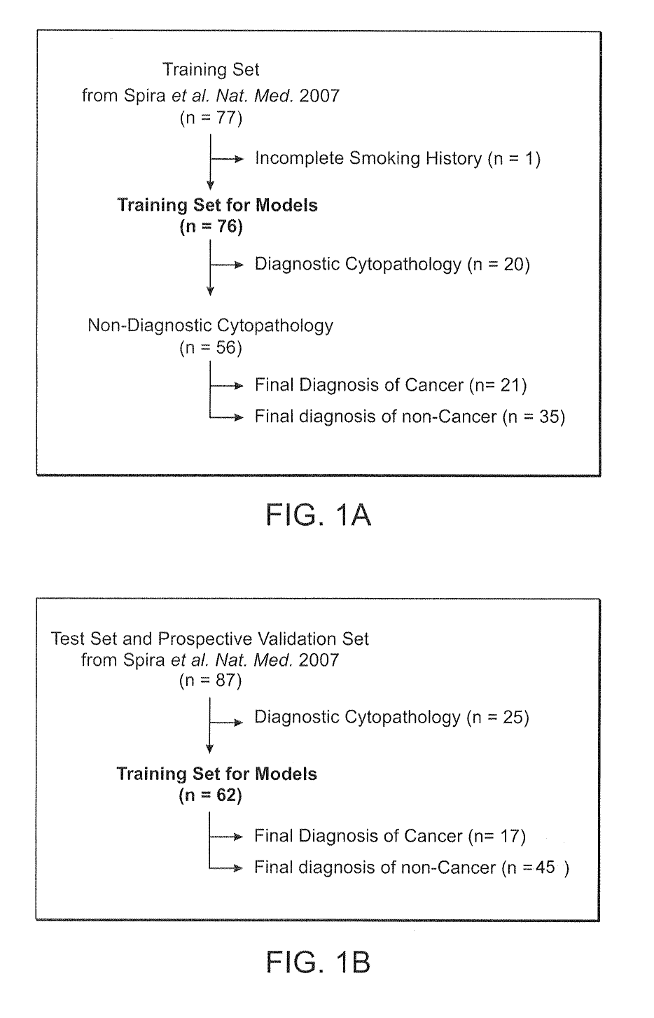

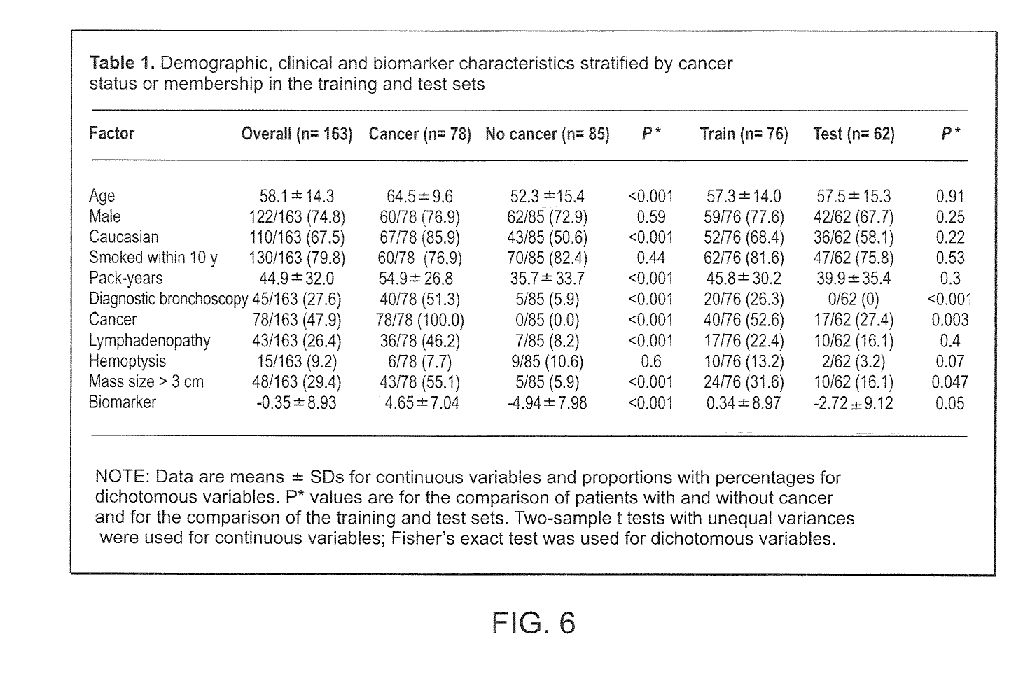

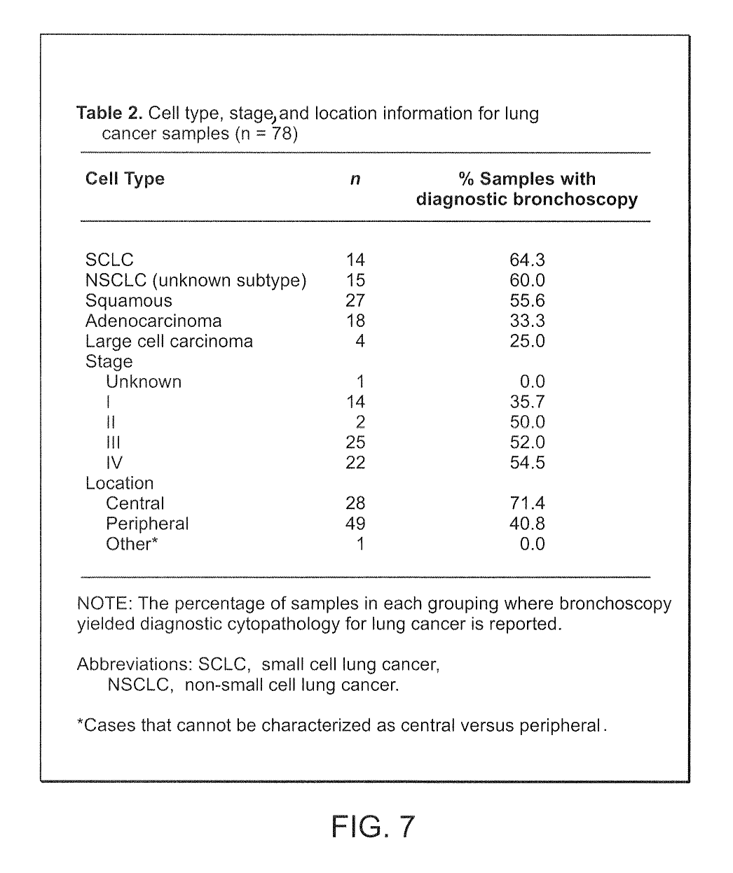

The present study cohort consists of patients who participated in a previous study to develop the large airway gene expression biomarker (Spira et al., Nat Med 13:361-366 (2007)). In that study, current and former smokers undergoing flexible bronchoscopy for clinical suspicion of lung cancer were recruited at four tertiary medical centers between January 2003 and April 2005 as previously described (Spira et al., Nat Med 13:361-366 (2007)). All subjects were >21 years of age and had no contraindications to flexible bronchoscopy. Never smokers and subjects who only smoked cigars were excluded from the study. All subjects were followed after bronchoscopy until a final diagnosis of lung cancer or an alternative diagnosis was made (mean follow-up time, 52 days). One hundred twenty-nine subjects (60 smokers with lung cancer and 69 smokers without lung cancer) who achieved final diagnoses as of May 2005 and had high quality microarray data were included in the primary sample set. Seventy-seven of these samples were randomly assigned to the training set. The training set for the current study (n=76) excluded one of these training set samples due to incomplete smoking history (FIG. 1A-1B). After completion of the primary study, a second set of samples (n=35) was collected prospectively from smokers undergoing flexible bronchoscopy for clinical suspicion of lung cancer at five medical centers between June 2005 and January 2006. Inclusion and exclusion criteria were identical to the primary sample set. The test set samples in the current study (n=87) combined both the remaining samples from the primary sample set (n=52) and this prospective test set (n=35), but the test set was limited to the subset of patients that did not have a definitive diagnosis following the bronchoscopy (n=62), as is shown in FIG. 1A-1B and described in more detail below. Demographic information on all subjects is detailed in Table 1 (FIG. 6) and information about the cell type, stage, and location of the lung tumors (n=78) in the study cohort is shown in Table 2 (FIG. 7). The study was approved by the Institutional Review Boards of the five medical centers at which patients were recruited (Boston University Medical Center, Boston, MA; Boston Veterans Administration, West Roxbury, Mass.; Lahey Clinic, Burlington, Mass.; St. James's Hospital, Dublin, Ireland; and St. Elizabeth's Medical Center, Boston, Mass.) and all participants provided written informed consent.

### Large Airway Gene Expression Biomarker for Lung Cancer

Using the Affymetrix HG-U133A microarray, a gene expression biomarker for lung cancer was previously developed using gene expression profiles in cytologically normal large airway epithelial cells collected from brushing the right mainstem bronchus of smokers undergoing bronchoscopy for suspicion of lung cancer (Gene Expression Omnibus accession no. GSE4115; Spira et al., Nat Med 13:361-366 (2007)). The biomarker was developed using the training set of the current study (n=76) with the addition of one additional sample that did not have a complete smoking history (FIG. 1A-1B). The biomarker was constructed from the expression levels of 80 probe sets (72 unique genes, 7 unannotated transcripts, and 1 redundant probe set) using the weighted-voting algorithm (Golub et al., Science 286:531-537 (1999)) that combines these expression levels into a biomarker score. A positive score is predictive of cancer and a negative score is predictive of no cancer.

In this study, the biomarker score was used as a starting point for the following statistical analyses: (a) building three logistic regression models to determine the likelihood of lung cancer using the clinical risk factors alone, the biomarker alone, or the likelihood of cancer using the clinical risk factors and biomarkers combined; (b) comparison of predictive values on a test set of patients not used in the initial model building phase; and (c) comparison of the clinical models with assessments made by expert clinicians.

### Construction of Logistic Regression Models

Logistic regression models to quantify the probability of a patient having lung cancer were generated using the training set samples (n=76). This training set included patients who had cytopathology findings that confirmed a diagnosis of either lung cancer or alternate noncancer pathology. Patients with diagnostic bronchoscopies were included in the training set to maximize the number of samples and because exclusion of these samples was unnecessary to develop models capable of accurately predicting the lung cancer status of patients with nondiagnostic bronchoscopies.

For the clinical and clinicogenomic models, the available clinical variables (Table 1; FIG. 6) included age, pack-years of smoking, and the following dichotomous variables; gender (male, 1: female, 0), race (1, Caucasian; 0, otherwise), hemoptysis (1, presence; 0, otherwise), lymphadenopathy (1, mediastinal or hilar lymph nodes 0.1 cm on computed tomography chest scan; 0, otherwise), and mass size (1, having a mass size >3 cm; 0, otherwise). Positron emission tomography scan information was only available for 15 patients and was not included in the model. Backward stepwise model selection using Akaike's information criterion (Akaike, IEEE Trans Automatic Control 19:716-723 (1974)) was used to select the optimal clinical model for the probability of a patient having lung cancer.

To create an integrated clinicogenomic model and determine the independence and magnitude of the contribution of the gene expression biomarker after adjusting for the effects of the clinical variables, the biomarker was first added to the optimal clinical model. The biomarker scores and all of the available clinical variables were then used with backward stepwise model selection by Akaike's information criterion to select the optimal model. Both approaches yielded the same combined model. To verify that the biomarker score performs similarly in logistic regression as in the weighted-voting prediction algorithm used in previous work (Spira et al., Nat Med 13:361-366 (2007)), the accuracy, sensitivity, specificity, positive predictive value, and negative predictive value were compared for the weighted-voting predictions and the predictions made by a logistic regression model that included only the biomarker score across the independent test samples.

### Comparison of Model Performance on Independent Patients

The performance of the logistic regression models (clinical, biomarker, and clinicogenomic) was initially evaluated on the subset of patients in the training set (n=76) in which the cytopathology of materials obtained at bronchoscopy was nondiagnostic (n=56; FIG. 1A-1B). We chose to focus on nondiagnostic bronchoscopies to specifically assess the utility of the gene expression biomarker and clinical parameters in the setting of patients that require further diagnostic evaluation for lung cancer. More importantly, we also tested the models in the nondiagnostic bronchoscopy test set (n=62; FIG. 1A-1B). For each of the models, patients that had a probability of lung cancer ≧0.5 were classified as having lung cancer, and patients with a probability <0.5 were classified as not having lung cancer. Receiver operating characteristics (ROC) curves were also used to compare the clinical model with the clinicogenomic model in the training set patients with nondiagnostic bronchoscopies, the independent test set, and combined set of all patients with nondiagnostic bronchoscopies (n=118). To assess whether or not two ROC curves based on the same set of samples were significantly different, methods developed for comparing ROC curves derived from the same cases were used (Hanley and McNeil, Radiology 143:29-36 (1982); Hanley and McNeil, Radiology 148:839-843 (1983)). To compare ROC curves based on different sample sets, a two-sample z test was used. The ROC curves serve as a common scale for evaluating the additional merit of variables added to the model because odds ratios for two different variables may not be comparable (Sullivan et al., J Natl Cancer Inst 93:1054-1061 (2001)). The accuracy, sensitivity, specificity, positive predictive value, and negative predicate value were also calculated across the independent test set for the clinical model, the biomarker model, and the clinicogenomic model.

### Subjective Clinical Assessment

Three independent pulmonary clinicians practicing at a tertiary medical center, blinded to the final diagnoses, evaluated each patient's clinical history at the time of the bronchoscopy. The history included, but was not limited to, age, smoking status, cumulative tobacco exposure, comorbidities, symptoms/signs, radiographic findings, and positron emission tomography scan results if available. Based on this information, the clinicians classified each patient into one of the three risk groups: low (<10% assessed probability of lung cancer), medium (10-50% assessed probability of lung cancer), and high (>50% assessed probability of lung cancer). The final subjective assignment for each subject was decided by choosing the median opinion. The inter-rater reliability for the clinical classification of patients' nondiagnostic bronchoscopies was significant, indicating that the level of agreement between the clinicians was greater then would be expected by chance as measured by the K statistic (K=0.57; P<0.001; ref. 28).

Comparison of Subjective Clinical Assessment with the Clinicogenomic Model

The sample size for building a comprehensive clinical model to predict the risk of having lung cancer was limited as was the scope of variables that were available for inclusion in the clinical and clinicogenomic models. We therefore sought to determine if the clinical model performs similarly to the subjective clinical assessment made by pulmonary specialists because this assessment is (a) “trained” on the large number of patients seen over each clinician's career and (b) considers all of the information contained within a patient's medical records. A Wilcoxon test was used to assess whether or not the clinical model-derived probability of having lung cancer varied between samples classified as low, medium, or high cancer risk by the clinicians.

### Statistical Analysis

All statistical analyses were conducted using R statistical software version 2.2.1.

### Results

### Evaluating the Gene Expression Biomarker as an Independent Predictor of Lung Cancer

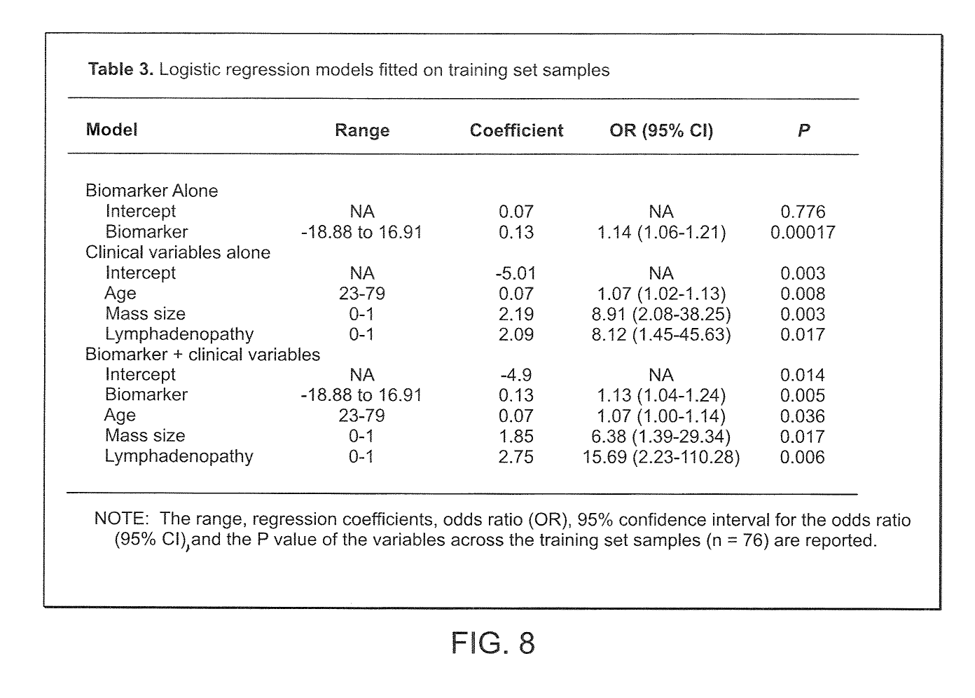

The demographic and clinical characteristics as well as the mean and SD for the biomarker scores stratified by cancer status and membership in the training or test sets are shown in Table 1 (FIG. 6). Age, race, pack-years of smoking, lymphadenopathy, mass size, and the biomarker score were significantly different (P<0.001) between patients with and without lung cancer. The test and training sets, however, were well balanced for the variables used in the analyses (although the incidence of having a mass size >3 cm was somewhat lower in the test set compared with the training set; P=0.047). Information about the cell type, stage, and location of the tumors in the cancer patients, as well as the fraction of diagnostic bronchoscopies for each subgroup, is shown in Table 2 (FIG. 7). Effect estimates and derived odds ratios for the variables in each of the three logistic regression models are shown in Table 3 (FIG. 8). We found that the optimal clinical model for this cohort did not include pack-years. This is likely due to the strong correlation between age and pack-years. The optimal clinical model did not include smoking status (former versus current smokers) regardless of how time since quitting was dichotomized. In addition, dichotomizing mass size using a threshold value of 2 cm (instead of 3 cm) produced clinical and clinicogenomic models with similar overall accuracy.

A logistic regression model describing the likelihood of having lung cancer derived from the biomarker score produced equivalent results to the weighted-voting algorithm predictions of lung cancer status previously (Postmus, Chest 128:16-18 (2005)), resulting in eight versus seven incorrect classifications, indicating that the biomarker score is an accurate way to model the original biomarker prediction algorithm in the clinicogenomic model. The biomarker score is a significant predictor of lung cancer likelihood both in the biomarker only model (P<0.001) and in the clinicogenomic model (P<0.005). In the clinicogenomic model, the coefficients of the clinical variables are largely unchanged from the clinical model, and the coefficient of the biomarker is largely unchanged from the biomarker only model. These data suggests that the gene expression biomarker and the clinical variables are independent predictors of lung cancer risk.

### Evaluating the Performance of the Clinicogenomlc Model

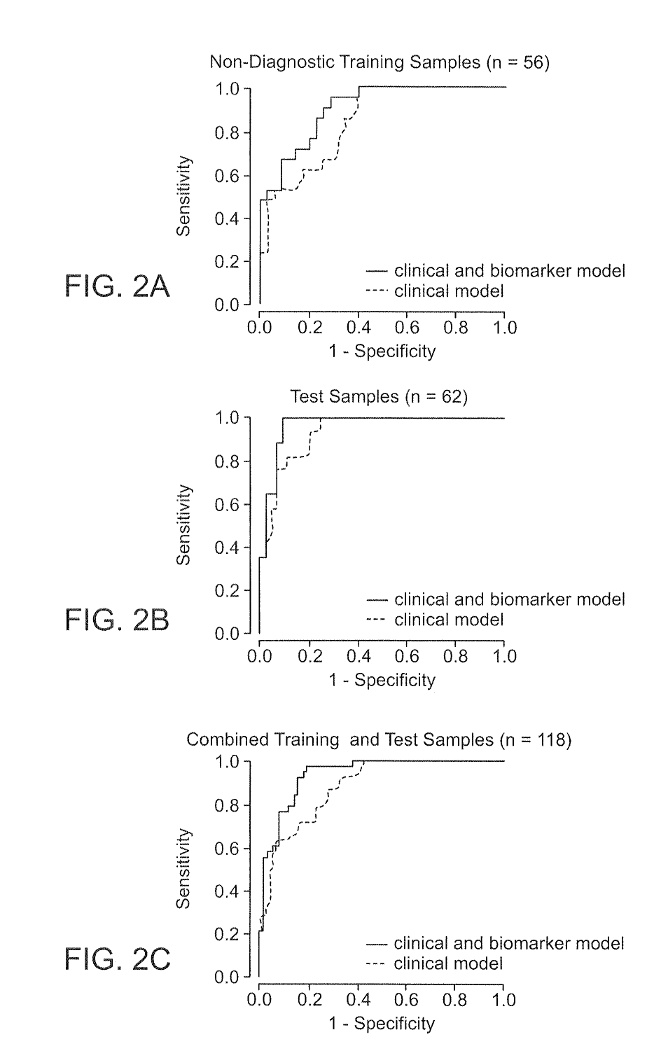

The three models were used to predict the cancer status subset of the training samples with nondiagnostic bronchoscopies (n=56), the independent test samples (n=62), and these two sets combined (n=118) ROC curves were used to compare the performance of the clinical model with that of the clinicogenomic model (FIG. 2A-2C). The clinicogenomic model had better performance than the clinical model in all three sample sets. Whereas this difference in performance does not reach statistical significance in the test set, when the training and test sets were combined, there was a significant difference in the area under the curve between the clinicogenomic and clinical models (P<0.05). The performance of the models in the training set samples does not seem to be any better than in the test set samples (P=0.25), for the difference in the area under the ROC curves; the area under the curve difference is 0.065; 95% confidence interval, −0.046 to 0.174). This suggests that the models do not overfit the training data and that it is therefore reasonable to combine the training and test sets to assess the significance of the difference in the performance of the clinical and clinicogenomic models.

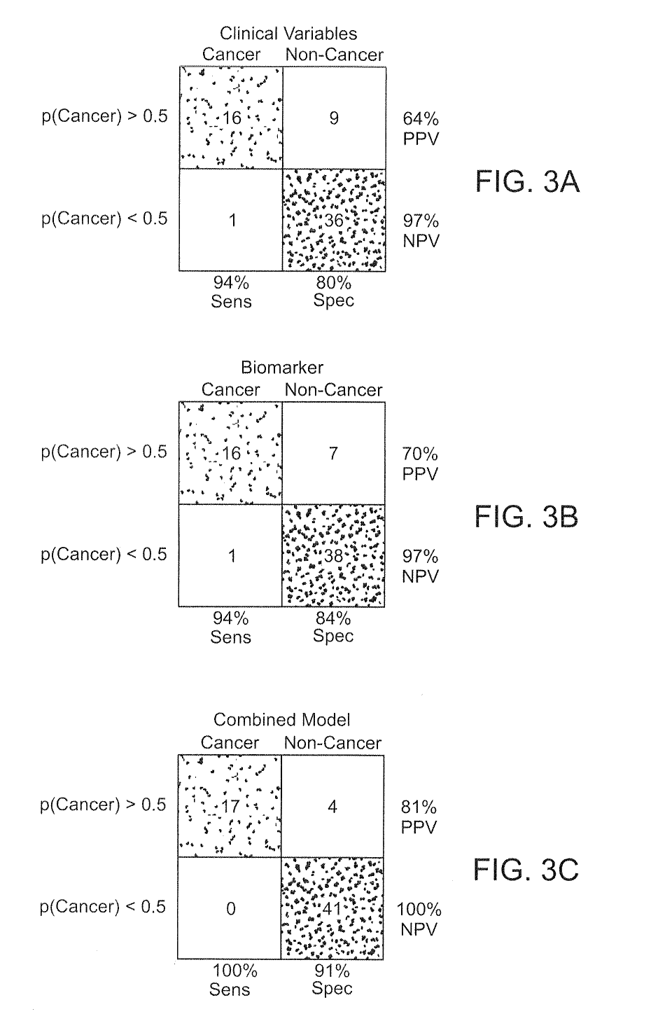

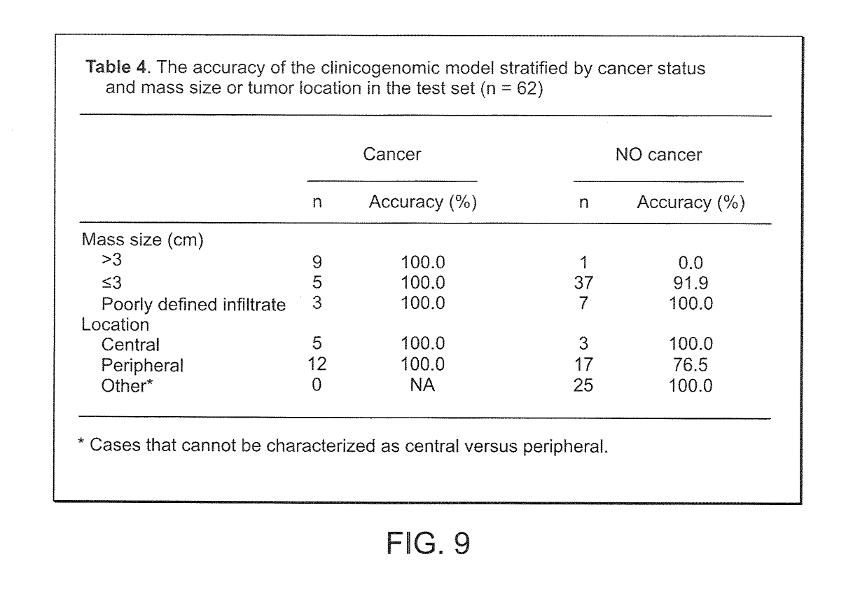

The sensitivity, specificity, positive predictive value, and negative predictive value for each of the three models were evaluated across the test set (FIG. 3A-3C). The combined clinicogenomic model increases the sensitivity and negative predictive value to 100% and results in higher specificity and positive predictive value compared with the other models. Cancer subjects with peripheral lesions were well represented I the test set (70.6%), and the clinicogenomic model was equally accurate among the peripheral or central lung tumors. The clinicogenomic model also accurately predicted lesions with a mass size <3 cm as well as poorly defined radiographic infiltrates in the test set (Table 4; FIG. 9). In addition, the performance of the clinical and clinicogenomic models does not seem to be specific to samples with nondiagnostic bronchoscopies because these models had sensitivities of 90% ad 95% on independent samples with diagnostic bronchoscopies (n=25). Finally, training the clinical and clinicogenomic models across only the training samples with nondiagnostic bronchoscopies (n=56) resulted in similar accuracies In the test set (82% and 91%, respectively) and a significant difference in the area under the ROC curves between the models (P<0.05).

Comparing the Clinicogenomic Model with the Clinical Subjective Assessment

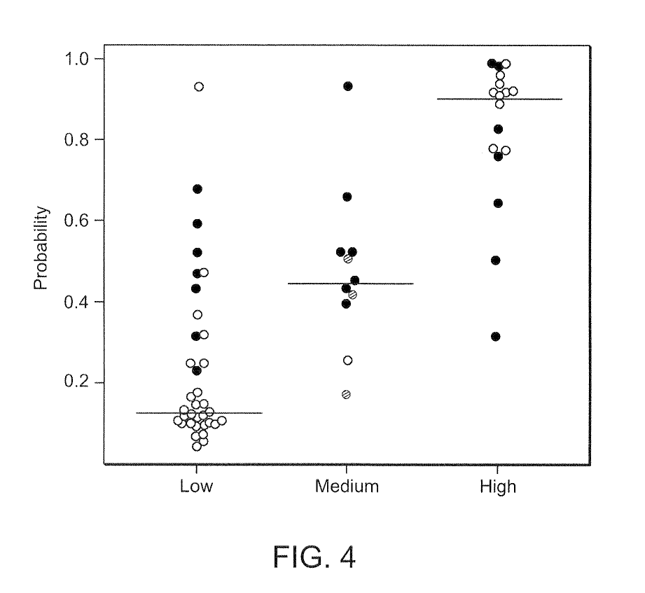

To evaluate whether or not the clinical model is comprehensive given the relatively small number of variables it contains, we assessed whether it correlates with the median subjective assessment of three pulmonary physicians. There was an association between the clinical model predictions and the clinical subjective assessment across the test set samples (FIG. 4). The clinical model probabilities were significantly different between the three physician-assessed risk groups (P<0.01).

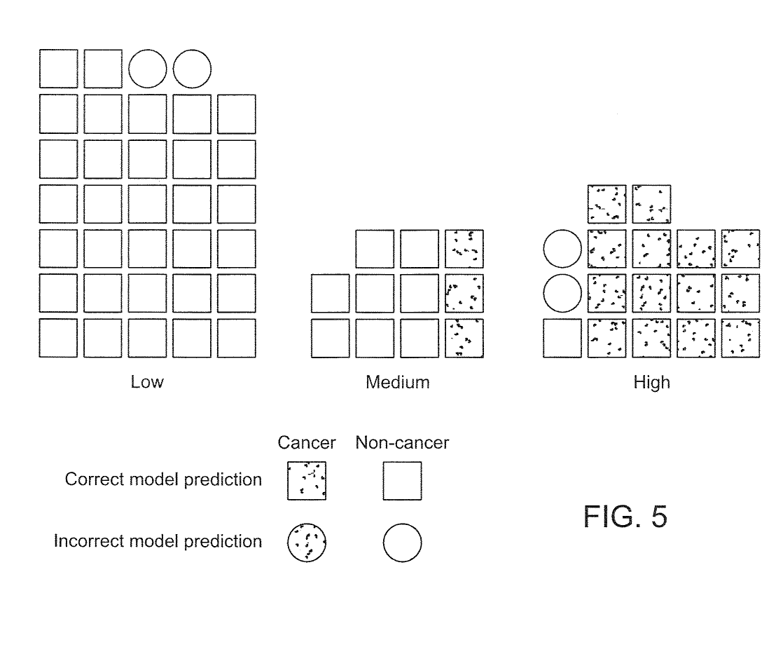

Given the association between the clinical model and subjective clinical assessment, we examined the predictions made by the clinicogenomic model stratified by cancer status and subjective clinical assessment category in the test set samples (FIG. 5). The physician's opinion is the most uncertain based on all the clinical data for the 11 samples in the medium risk category. The clinical model is able to classify 7 of the 11 samples correctly; however, the clinicogenomic model correctly classifies all 11 samples.

What is claimed is:

1. A method of aiding in the diagnosis of lung disease in a patient suspected of having lung disease, comprising:
analyzing two or more independent lung cancer-relevant diagnostic paradigms in a patient to be assessed; and
determining a composite classification of the patient as having lung disease or not having lung disease.

2. A method according to claim 1 wherein the lung disease is lung cancer.

3. A method according to claim 1 wherein the patient is a smoker or former smoker.

4. A method according to claim 1 wherein the patient has had an abnormal radiographic finding or a nondiagnostic bronchoscopy.

5. A method according to claim 1 wherein the two or more lung cancer-relevant diagnostic paradigms are selected from the group consisting of analyzing expression of one or more lung cancer-relevant genes in the patient, analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microPNAs.

6. A method according to claim 1 wherein the two or more lung cancer-relevant diagnostic paradigms comprise analyzing expression of one or more lung cancer-relevant genes in the patient.

7. A method according to claim 1 wherein the two or more lung cancer-relevant diagnostic paradigms comprise analyzing expression of one or more lung cancer-relevant genes in the patient and one or more lung cancer-relevant diagnostic paradigms selected from the group consisting of analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs.

8. A method according to claim 6 wherein the one or more lung cancer-relevant genes are all or a subset of the genes for which expression data is contained in Gene Expression Omnibus accession no GSE4115.

9. A method according to claim 7 wherein the one or more lung cancer-relevant genes are all or a subset of the genes for which expression data is contained in Gene Expression Omnibus accession no. GSE4115.

10. A method according to claim 1 wherein the two or more lung cancer-relevant diagnostic paradigms comprise analyzing expression of one or more lung cancer-relevant genes in the patient and analyzing one or more lung cancer-relevant clinical factors or variables of the patient.

11. A method according to claim 10 wherein the one or more lung cancer-relevant genes are all or a subset of the genes for which expression data is contained in Gene Expression Omnibus accession no. GSE4115.

12. A method of determining a follow up treatment regimen for a patient suspected of having lung cancer, comprising
analyzing two or more independent lung cancer-relevant diagnostic paradigms in a patient to be assessed, and classifying the patient as having cancer or not having cancer on the basis of the analysis,
wherein a patient classified as having cancer is selected for invasive testing and/or commencement of therapeutic regimen, and a patient classified as not having cancer is monitored without invasive testing or commencement of therapeutic regimen.

13. A method according to claim 12 wherein the patient is a smoker or former smoker.

14. A method according to claim 12 wherein the patient has had an abnormal radiographic finding or a nondiagnostic bronchoscopy.

15. A method according to claim 12 wherein the two or more lung cancer-relevant diagnostic paradigms are selected from the group consisting of analyzing expression of one or more lung cancer-relevant genes in the patient, analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs.

16. A method according to claim 12 wherein the two or more lung cancer-relevant diagnostic paradigms comprise analyzing expression of one or more lung cancer-relevant genes in the patient.

17. A method according to claim 12 wherein the two or more lung cancer-relevant diagnostic paradigms comprise analyzing expression of one or more lung cancer-relevant genes in the patient and one or more lung cancer-relevant diagnostic paradigms selected from the group consisting of analyzing one or more lung cancer-relevant clinical factors or variables of the patient, testing for the presence or absence of one or more lung cancer-relevant antibodies in the patient's blood, testing for the presence or absence of one or more lung cancer-relevant proteins in the patient's blood, and analyzing expression of one or more lung cancer-relevant microRNAs.

18. A method according to claim 16 wherein the one or more lung cancer-relevant genes are all or a subset of the genes for which expression data is contained in Gene Expression Omnibus accession no. GSE4115.

19. A method according to claim 12 wherein the two or more lung cancer-relevant diagnostic paradigms comprise analyzing expression of one or more lung cancer-relevant genes in the patient and analyzing one or more lung cancer-relevant clinical factors or variables of the patient.

20. A method of aiding in the diagnosis of lung cancer in a patient suspected of having lung cancer, comprising:
obtaining a biological sample from the patient and analyzing expression of one or more lung cancer-relevant genes in the sample, wherein the one or more lung cancer-relevant genes are all or a subset of the genes for which expression data is contained in Gene Expression Omnibus accession no. GSE4115;
analyzing one or more lung cancer-relevant clinical factors or variables of the patient; and
determining a composite classification of the patient as having cancer or not having cancer.
